(19) United States
(12) Patent Application Publication
(10) Pub. No.: US 2007/0270966 A1
(43) Pub. Date: Nov. 22, 2007

(54) Spinal implant loading block with multiple orientations

(75) Inventor: Richard Evan Chen, Memphis, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: SDGI Holdings, Inc.

(21) Appl. No.: 11/414,833

(22) Filed: Apr. 28, 2006

(51) Int. Cl. A61F 2/44

(52) U.S. Cl. 623/17.11

(57) ABSTRACT

Spinal implant systems, apparatus, and methods of use are provided. One such apparatus for holding an implantable spinal device includes a base having a generally flat first surface and a second surface spaced apart from the first surface. The base has a third surface between the first and second surfaces and generally orthogonal to at least one of the first and second surfaces. The base further includes a fourth surface spaced apart from the third surface. At least one cavity is provided in the base, with the cavity being accessible through the base second and fourth surfaces, and adapted to receive the implantable spinal device. In some embodiments, the spinal implant has an opening accessible through the fourth surface when the spinal device is positioned in the cavity. In this manner, bony growth-promoting substances may be inserted into the implant when the implant is positioned in the apparatus.

### BACKGROUND OF THE INVENTION

The present invention relates generally to orthopedic implants used for correction of spinal injuries or deformities, and more specifically, but not exclusively, to holding apparatus, systems and methods for preparing spinal implants for insertion into a patient.

In the field of spinal surgery, it is known to place implants into vertebrae for a number of reasons, including (a) to correct an abnormal curvature of the spine, including a scoliotic curvature, (b) to maintain appropriate spacing and provide support to broken or otherwise injured vertebrae, and (c) to perform other therapies on the spinal column.

Some treatments involve the removal of a disk, distraction of the disk space, and the insertion of an interbody device between two adjacent vertebrae. In some cases, it may be desirable to load the interbody device with bone or other material to promote bony ingrowth or fusion. To do so, the surgeon often must remove the implant from its sterile packaging, find a flat, sterile surface on which to place the implant, and thereafter pack the implant with the desired material, all while trying to keep the implant from sliding around or falling to the floor. Improvements are desired.

### SUMMARY OF THE INVENTION

The present invention provides spinal implant systems, apparatus, and methods of use. In one embodiment, an apparatus for holding an implantable spinal device according to the present invention includes a base having a generally flat first surface and a second surface spaced apart from the first surface to define a base thickness. The base has a third surface between the first and second surfaces and generally orthogonal to at least one of the first and second surfaces. The base further includes a fourth surface spaced apart from the third surface. At least one cavity is provided in the base, with the cavity being accessible through the base second and fourth surfaces, and adapted to receive the implantable spinal device. In some embodiments, the spinal implant has an opening accessible through the fourth surface when the spinal device is positioned in the cavity. In this manner, bony growth-promoting substances may be inserted into the implant when the implant is positioned in the apparatus.

In one aspect, the third surface is generally orthogonal to both the first and second surfaces. In another aspect, the base includes at least three spaced apart cavities, with each of the cavities accessible through the second and fourth surfaces, and inaccessible through the first and third surfaces. In alternative embodiments, at least two of the three cavities have a different size, a different width, and/or a different depth. Further, the cavity may have a shape which is adapted to frictionally engage the implantable spinal device. In still another aspect, the cavity further includes a lip extending over a portion of the cavity and generally aligned with the second surface. The lip may, for example, operate to help maintain the implant in the cavity.

The present invention further provides spinal implant systems. In one such embodiment, the system includes a loading block having a generally flat first surface and a second surface spaced apart from the first surface. The loading block has a third surface between the first and second surfaces, and a fourth surface spaced apart from the third surface. The loading block further includes a chamber disposed therein, with the chamber being open through the base second and fourth surfaces. A spinal implant is disposed in the chamber. The implant has a cavity therein for receiving a bone growth-promoting substance, with the cavity generally aligned with the chamber opening through the fourth surface.

In one aspect, the loading block and spinal implant are adapted to be sterilized in a same process. The system may further comprise additional instruments, including, a packing instrument adapted for packing the bone growth-promoting substance in the implant cavity when the implant is disposed in the chamber, and an implant removal instrument adapted to remove the spinal implant from the chamber. The spinal implant, in one aspect, includes a tool engaging recess generally aligned with the chamber opening through the second surface. The recess is adapted to engage the removal instrument.

The present invention further provides methods, including methods of preparing a spinal implant for insertion in a patient. In one such embodiment, the method includes providing a generally rectangular loading block having a chamber therein, inserting a spinal implant into the chamber, and sterilizing the combined loading block and spinal implant. The method includes positioning the loading block on a first side thereof and inserting a material, such as a bone growth-promoting substance, into the spinal implant. The loading block is positioned on a second side thereof and the spinal implant is engaged with an instrument adapted for removing the spinal implant from the loading block.

In some aspects, the first and second loading block sides are generally orthogonal. The chamber may define an opening through third and fourth sides of the loading block generally opposite the first and second sides, respectively. In one aspect, the material is inserted through the chamber opening in the third side for packing into the spinal implant. Engagement of the spinal implant may occur through the chamber opening in the fourth side. In some aspects, the engagement of the spinal implant includes threadingly engaging the spinal implant with the instrument. In other aspects, the spinal implant is removed from the loading block through the chamber opening in the fourth side. The instrument used to remove the implant from the loading block, in some embodiments, is the same instrument used to insert the spinal implant into a patient.

Other features and advantages of the invention will appear from the following description in which the preferred embodiment has been set forth in detail in conjunction with the accompanying drawings.

### BRIEF DESCRIPTION OF THE DRAWINGS

FIG. 1 shows an overall view of an apparatus for holding a spinal implant according to an embodiment of the present invention;

FIG. 2 is an overall view of a spinal implant for use with apparatus and systems of the present invention;

FIG. 3 shows an overall view of an apparatus for holding a spinal implant according to another embodiment of the present invention;

FIGS. 4A and 4B show spinal implant systems according to embodiments of the present invention; and

FIG. 5 is a simplified flow chart of a method according to an embodiment of the present invention.

### DESCRIPTION OF THE PREFERRED EMBODIMENTS

Reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of embodiments of the invention as illustrated therein, being contemplated as would normally occur to one skilled in the art to which the invention relates.

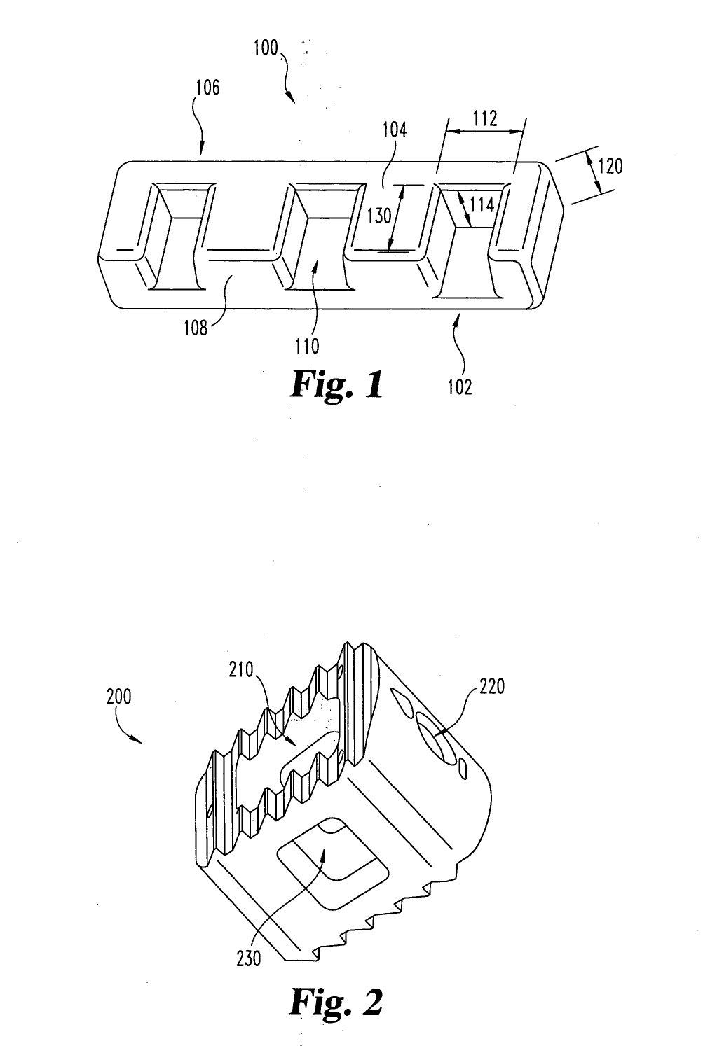

Turning now to FIG. 1, a loading block 100 according to an embodiment of the present invention will be described. In the depicted embodiment, loading block 100 has a first side 102 and a second side 104 spaced apart from first side 102. Spaced apart first and second sides 102, 104 define a loading block thickness 120. In one embodiment, first and second sides 102, 104 are generally parallel to one another, resulting in a generally uniform thickness 120. In alternative embodiments, first and second sides 102, 104 are not parallel. In the depicted embodiment, loading block 100 includes a third side 106 and a fourth side 108. Again, in one embodiment, third and fourth sides 106, 108 are generally parallel to one another, although in alternative embodiments they are not parallel. In a preferred embodiment, third side 106 is generally orthogonal to first side 102. Third side 106 also may be generally orthogonal to second side 104.

Loading block 100 depicted in FIG. 1 has a generally rectangular shape. Other overall shapes of loading block 100 also fall within the scope of the present invention. For example, in one embodiment second side 104 is a generally flat surface, or a curved surface, that is spaced a varying distance from first side 102. This can be accomplished, for example, by having third and fourth sides 106, 108 differ in length, or by having first and second sides 102, 104 come together so that block 100 does not have a fourth side 108. In other embodiments, fourth side 108 is shorter or longer than thickness 120. In this manner, loading block 100 may have a generally trapezoidal or other cross-sectional shape, compared to the generally rectangular shape shown in FIG. 1.

As best seen in FIG. 1, loading block 100 has one or more chambers or cavities 110. Three spaced apart cavities 110 are shown, with each cavity 110 having a different overall size. In alternative embodiments of the present invention, loading block 100 has a greater or smaller numbers of cavities 110. Block 100 also may have cavities 110 of varying size, or a uniform size. In the depicted embodiment, cavities 110 vary in a width 112 and/or a depth 114. For example, in one embodiment, loading block 100 has three cavities 110 with different dimensions. A first cavity 110 is generally eleven millimeters (11 mm) wide and eleven millimeters (11 mm) deep. A second cavity 110 is fourteen millimeters (14 mm) wide and eleven millimeters (11 mm) deep. A third cavity 110 is fourteen millimeters (14 mm) in both depth and width. The particular sizes of cavities 110 will vary within the scope of the present invention, depending in part on the size of implant to be used with loading block 100.

Loading block 100 preferably comprises a material which lends itself to being sterilized. For example, loading block 100 may comprise a metal, ceramic, polymer, or the like. In this manner, loading block 100 may be used in sterile procedures and/or with implantable devices requiring a high degree of cleanliness. Further, in some embodiments, loading block 100 can be preloaded with an implantable medical device and sterilized therewith. As a result, the implant and loading block 100 can be delivered to the surgical site, operating room or the like, in a sterile condition.

In one embodiment, loading block 100, and more preferably cavities 110, are adapted to receive a medical implant 200 such as the spinal implant 200 shown in FIG. 2. Implant 200 is adapted to be inserted in a patient, preferably between two adjacent vertebrae. Implant 200 may be useful for promoting fusion between the two vertebrae, as well as providing support to the adjacent vertebrae normally provided by a disc. As shown, implant 200 includes a cavity or opening 210 which provides access to the middle or center portion of implant 200. Opposing surfaces of implant 200 are open, allowing access to cavity 210. This may be useful, for example, to allow bony growth therethrough subsequent to a bone fusion procedure. Implant 200 further includes a recess 220 or other means for engaging an instrument as further described below. In one embodiment, recess 220 is a threaded recess adapted to engage a threaded instrument.

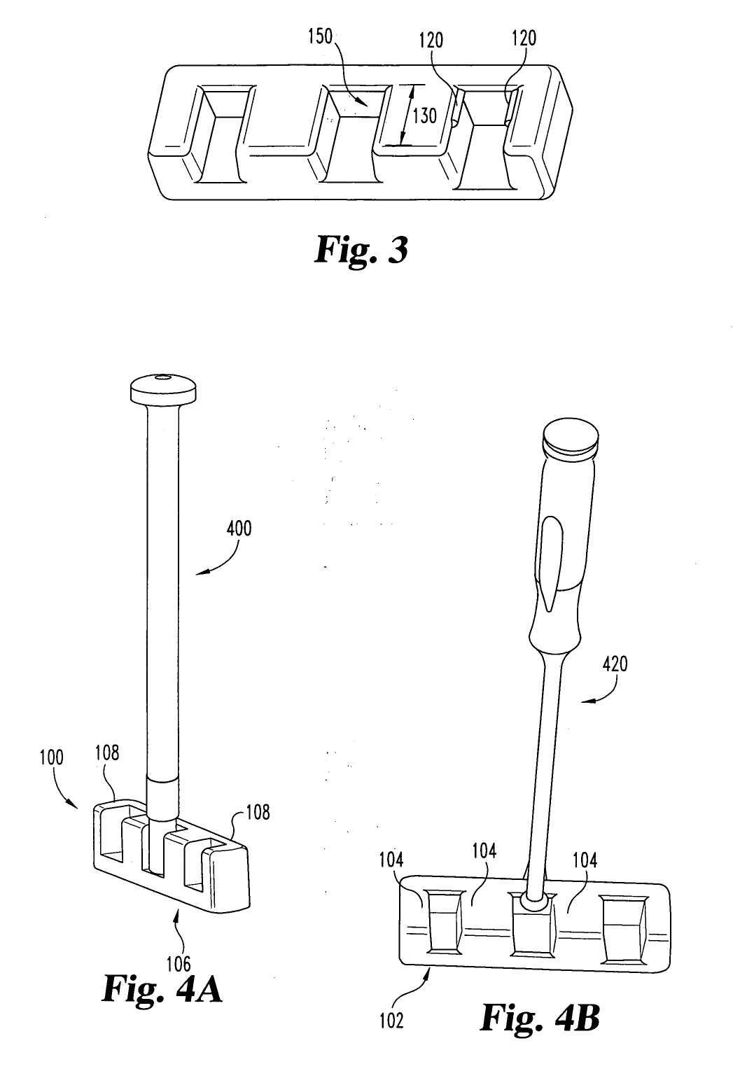

In one embodiment of the present invention, implant 200 is placed in cavity 110 of loading block 100. Preferably, the dimensions of at least one cavity 110 generally corresponds to the outer dimensions of implant 200. For example, an implant having an 11 mm by 11 mm width and depth may be positioned in a similarly dimensioned cavity 110. In one embodiment, one or more walls of cavity 110 acts to frictionally engage corresponding side surfaces of implant 200. In this manner, cavity 110 holds implant 200 therein to avoid unwanted or premature removal of implant 200. In an alternative embodiment, one or more cavities 110 include one or more lips 120. In the embodiment shown in FIG. 3, two lips 120 extend over cavity 110 along at least a portion of a length 130 of cavity 110. In this manner, lips 120 operate to help maintain implant 200 within cavity 110. In one embodiment, lips 120 extend along only a portion of length 130 as shown in FIG. 3. Alternatively, lips 120 may extend the full length 130 of cavity 110. In another embodiment, only a single lip 120 extends along some or all of length 130 of cavity 110. In some of these embodiments, it may be desirable to slide implant 200 into and/or out of cavity 110 using the opening through fourth side 108 of loading block 100. In one embodiment, implant 200 is slid into cavity 110 through the opening in the fourth side of 108 and slid until lip 120 engages an upper surface of implant 200. In this manner, implant 200 is sufficiently held in place by cavity 110 walls and/or lip 120. In one embodiment, lip 120 lies generally in a same plane as second side 104.

One advantage of the present invention involves loading block 100 having cavities 110 which allow access thereto from two sides of loading block 100. For example, as shown in FIGS. 1 and 3, cavities 110 are open through second side 104 and fourth side 108 of loading block 100. This feature will be particularly useful, for example, to allow a same loading block 100 to hold implant 200 during multiple process steps performed prior to and during surgery. For example, as shown in FIG. 4A, loading block 100 is positioned to rest on third side 106, which exposes fourth side 108 to a packing instrument 400. In one embodiment, packing instrument 400 is a trephine. Packing instrument 400 may be used, for example, to pack a bone growth-promoting substance into implant 200, when implant 200 is disposed within cavity 110. In the configuration shown in FIG. 4A, cavity/opening 210 of implant 200 is generally aligned with fourth side 108 of loading block 100. In this manner, a bone growth-promoting substance may be packed into implant 200 through opening 210. Further, the opposite side of implant 200 rests against a wall 150 of cavity 210. Wall 150 provides a firm surface against which the bone growth-promoting material may be packed or compressed for embodiments in which implant opening 210 extends through implant 200.

Once implant 200 is sufficiently packed, in one embodiment loading block 100 is placed on first side 102. Placing first side 102 on a firm surface, such as a tray or surgical table, provides a stable environment for implant 200 during subsequent processes. In doing so, second side 104 is exposed to, for example, a tool or instrument 420 adapted for removing implant 200 from loading block 100. Instrument 420 may engage recess 220 in implant 200 to remove implant 200 from loading block 100. In one embodiment, the removal of implant 200 from loading block 100 includes threadingly engaging instrument 420 into recess 200, and sliding implant 200 along cavity 100 until implant 200 passes out of cavity 110 through the opening in fourth side 108. This may be useful, for example, in embodiments utilizing the loading block 100 shown in FIG. 3 comprising lip(s) 120. Alternatively, implant 200 may be slid along cavity 100 until it is no longer engaging or disposed under lip(s) 120. Implant 200 then can be removed from loading block 100 through second surface 104. In an alternative embodiment, the fit or conformity in shape between implant 200 and cavity 110 is such to permit removal of implant 200 directly through second side 104.

In this manner, a single loading block 100 may be used for delivering a sterile implant 200 to the operating site, for holding implant 200 while bone growth-promoting material (or other material, medicine, etc.) is inserted into implant 200, and/or for providing a stable platform to hold implant 200 while implant 200 is coupled to an instrument (such as instrument 420). In one embodiment, instrument 420 not only removes implant 200 from loading block 100, but also is the instrument or a portion of the instrument used to insert implant 200 into the patient.

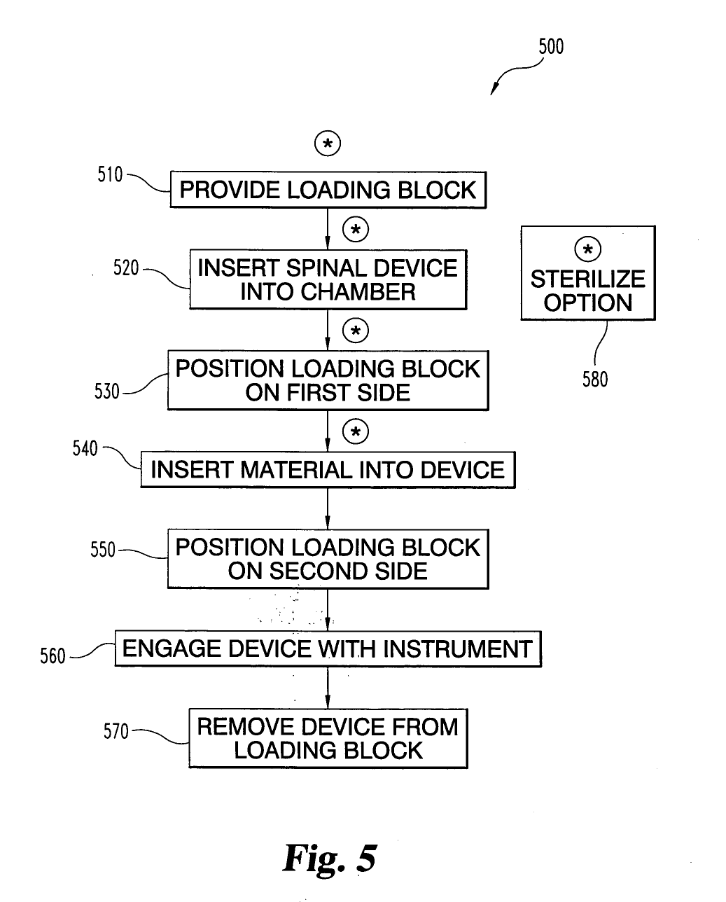

Turning now to FIG. 5, one embodiment of a method according to the present invention will be described. Method 500 includes providing a loading block (Block 510). For example, the loading block may be any loading block 100 according to embodiments of the present invention. Method 500 includes inserting a medical device or implant into a chamber or cavity in loading block (Block 520), positioning the loading block on a first side (Block 530) and inserting a material into the medical device (Block 540). Again, the material can comprise a bone growth-promoting substance, a medicine, or the like. Method 500 includes positioning the loading block on a second side thereof (Block 550), and engaging the medical device with a tool or instrument (Block 560). The method further includes removing the medical device from the loading block (Block 570).

As shown in FIG. 5, method 500 provides one or more opportunities to sterilize the loading block and/or medical device. Sterilization may occur, for example, prior to providing loading block. In this manner, the manufacture of loading block 100 and/or implant 200 may include desired sterilization procedures thereof, and allow for the deliver of a combined sterilized loading block 100/implant 200 to the surgeon. Alternatively, or in addition, sterilization may occur after loading block 100 has been provided, but prior to insertion of medical device 200 into loading block chamber 110. This may occur, for example, when loading block 100 and medical device 200 are provided separately and sterilized prior to completing the subsequent portions of method 500. In still another embodiment, the sterilization occurs after the medical device is inserted into loading block 100, and either before or after positioning the loading block on a first side thereof. Alternative methods of the present invention may include additional sterilization steps, such as inserting or rinsing the combined loading block/spinal implant in sterilized solution.

While the above description generally describes embodiments of the present invention, it would be appreciated by those skilled in the art that several alternatives exist within the scope of the present invention. For example, implant 200 depicted in FIG. 2 represents one of a wide variety of implants for use with loading block 100 of the present invention. For example, implant 200 is depicted with lateral ports 230 which may be absent from alternative embodiments of implants 200. Further, recess 220 provides one of several means for engaging a tool or instrument within the scope of the present invention. For example, recess 220 may comprise a threaded recess, a detent having a lip or other extension disposed therein which engages a corresponding ridge or lip in the distal tip of instrument 420, or the like. Instrument 420 may be a stab and grab type of instrument which has a compressible tip which is compressed prior to insertion into recess 220 and thereafter allowed to expand to engage the side walls of recess 220. In this manner, implant 200 may be removed or otherwise manipulated within loading block 100.

Bone growth-promoting substances may include a wide range of materials within the scope of the present invention. For example, the material may include an allograft, an autograft, bone morphogenic protein (BMP), as well as a product sold under the trademark INFUSE™ by Medtronic Sofamor Danek, Inc. Other bone growth-promoting substances, proteins, organic materials, inorganic materials and the like also may be used. In one embodiment, loading block 100 comprises a metal, a ceramic, a polymer, or the like, which is capable of being sterilized through using known or future sterilization processing. The sterilization may include, for example, sterilized solutions, autoclaves, or the like.

Components of the described embodiments, including loading block 100 may be made from a variety of materials compatible for use with the human body, including without limitation metals (e.g., titanium, nitinol, stainless steel), ceramics, polyethylene, PEEK, and other materials.

Having described several embodiments, it will be recognized by those skilled in the art that various modifications, alternative constructions, and equivalents may be used without departing from the spirit of the invention. Accordingly, the above description should not be taken as limiting of the scope of the present invention.

What is claimed is:

1. An apparatus for holding an implantable spinal device, the apparatus comprising:
a base having a generally flat first surface and a second surface spaced apart from the first surface to define a base thickness, the base having a third surface between the first and second surfaces and generally orthogonal to at least one of the first and second surfaces, the base further including a fourth surface spaced apart from the third surface;
wherein the base comprises at least one cavity disposed therein, the cavity being accessible through the base second and fourth surfaces; and
wherein the cavity is adapted to receive the implantable spinal device.

2. The apparatus as in claim 1 wherein the third surface is generally orthogonal to both the first and second surfaces.

3. The apparatus as in claim 1 wherein the base comprises at least three spaced apart cavities, each of the cavities accessible through the second and fourth surfaces, and inaccessible through the first and third surfaces.

4. The apparatus as in claim 3 wherein at least two of the at least three spaced apart cavities have a different size.

5. The apparatus as in claim 3 wherein at least two of the at least three spaced apart cavities have a different width.

6. The apparatus as in claim 3 wherein at least two of the at least three spaced apart cavities have a different depth.

7. The apparatus as in claim 1 wherein the at least one cavity has a shape which is adapted to frictionally engage the implantable spinal device.

8. The apparatus as in claim 1 wherein the at least one cavity further comprises a lip extending over a portion of the cavity and generally aligned with the second surface.

9. The apparatus as in claim 1 wherein the spinal device has at least one opening disposed therein, the opening accessible through the fourth surface when the spinal device is positioned in the at least one cavity.

10. A spinal implant system, comprising:
a loading block having a generally flat first surface and a second surface spaced apart from the first surface, the loading block having a third surface between the first and second surfaces, and a fourth surface spaced apart from the third surface, the loading block further comprising a chamber disposed therein, the chamber being open through the base second and fourth surfaces; and
a spinal implant disposed in the chamber, the implant having a cavity therein for receiving a bone-growth promoting substance, the cavity generally aligned with the chamber opening through the fourth surface.

11. The system as in claim 10 wherein the loading block and spinal implant are adapted to be sterilized in a same process.

12. The system as in claim 10 further comprising a packing instrument adapted for packing the bone-growth promoting substance in the implant cavity when the implant is disposed in the chamber.

13. The system as in claim 10 further comprising an implant removal instrument adapted to remove the spinal implant from the chamber.

14. The system as in claim 13 wherein the spinal implant further comprises a tool engaging recess, the recess generally aligned with the chamber opening through the second surface, the recess adapted to engage the removal instrument.

15. A method of preparing a spinal implant for insertion in a patient, the method comprising:
providing a generally rectangular loading block having a chamber therein;
inserting a spinal implant into the chamber;
sterilizing the combined loading block and spinal implant;
positioning the loading block on a first side thereof, and inserting a material into the spinal implant;
positioning the loading block on a second side thereof, and engaging the spinal implant with an instrument adapted for removing the spinal implant from the loading block.

16. The method as in claim 15 wherein the material comprises a bone growth-promoting substance.

17. The method as in claim 15 wherein the first and second loading block sides are generally orthogonal.

18. The method as in claim 15 wherein the chamber defines an opening through third and fourth sides of the loading block generally opposite the first and second sides, respectively.

19. The method as in claim 18 wherein the inserting of the material comprises packing the spinal implant with the material through the chamber opening in the third side.

20. The method as in claim 18 wherein the engaging of the spinal implant comprises engaging the spinal implant through the chamber opening in the fourth side.

21. The method as in claim 18 further comprising removing the spinal implant from the loading block through the chamber opening in the fourth side.

22. The method as in claim 15 wherein the engaging of the spinal implant comprises threadingly engaging the spinal implant with the instrument.

23. The method as in claim 22 wherein the instrument is further adapted to insert the spinal implant into a patient.
